(19) United States
(12) Patent Application Publication
(10) Pub. No.: US 2026/0167958 A1
(43) Pub. Date: Jun. 18, 2026

(54) USE OF SRSF1 IN PREPARATION OF MEDICAMENT FOR PROPHYLAXIS AND/OR TREATMENT OF HEART FAILURE WITH PRESERVED EJECTION FRACTION

(71) Applicant: Beijing Friendship Hospital, Capital Medical University, Beijing (CN)

(72) Inventors: Chunmei CAO, Beijing (CN); Jing GUO, Beijing (CN); Kun ZHU, Beijing (CN); Jingchen LI, Beijing (CN); Rilei DAI, Beijing (CN)

(21) Appl. No.: 19/035,225

(22) Filed: Jan. 23, 2025

(30) Foreign Application Priority Data
Dec. 17, 2024 (CN) 202411859598.X

(51) Int. Cl. C12N 15/113; A61P 9/04; C07K 16/18

(52) U.S. Cl. CPC C12N 15/113; A61P 9/04; C07K 16/18; C12N 2310/14; C12N 2310/531

(57) ABSTRACT

Provided is use of serine/arginine splicing factor 1 (SRSF1) in preparation of a medicament for prophylaxis and/or treatment of heart failure with preserved ejection fraction (HFpEF), which belongs to the field of biomedical technology. The SRSF1 is identified as a key target for preparing a medicament for the prophylaxis and/or treatment of HFpEF. Animal modeling and cellular level experiment has demonstrated that SRSF1 is involved in regulation of the pathological process of the disease. In vivo modeling experiment in SRSF1 endothelial cell-specific knockout mice indicates that inhibiting SRSF1 expression improves symptoms such as diastolic dysfunction and hypertension, alleviates pulmonary edema, and inhibits myocardial fibrosis and remodeling. Moreover, suppressing SRSF1 expression improves abnormal glucose/lipid metabolism and inhibits the endothelial-to-mesenchymal transition process. This application opens a new path for drug screening and provides a new target and a method for the treatment of HFpEF, holding important medical significance and value.

### CROSS REFERENCE TO RELATED APPLICATION

This patent application claims the benefit and priority of Chinese Patent Application No. 202411859598.X filed with the China National Intellectual Property Administration on Dec. 17, 2024, the disclosure of which is incorporated by reference herein in its entirety as part of the present application.

### REFERENCE TO SEQUENCE LISTING

A computer readable XML file entitled “GWP20241107484_seqlist”, that was created on Jan. 3, 2025, with a file size of about 3,144 bytes, contains the sequence listing for this application, has been filed with this application, and is hereby incorporated by reference in its entirety.

### TECHNICAL FIELD

The present disclosure relates to the field of biomedical technology, and in particular, to use of serine/arginine splicing factor 1 (SRSF1) in preparation of a medicament for prophylaxis and/or treatment of heart failure with preserved ejection fraction (HFpEF).

### BACKGROUND

Heart failure (HF), as the end stage of a plurality of cardiovascular diseases, has been showing a yearly increase in both its morbidity and mortality. According to epidemiological data, heart failure with preserved ejection fraction (HFpEF) accounts for 50% of the total number of HF, and has become the most common category of HF. It is particularly noteworthy that this proportion of HFpEF is expected to increase, especially given the continuous extension of human life expectancy and the increasing incidence and normalization of obesity and diabetes. Importantly, HFpEF not only has a high incidence and a serious aging trend, but also has a high mortality. It is also susceptible to various complications, bringing a very heavy burden to the health and life of patients.

In clinical practice, HFpEF is defined as a condition characterized by impaired ventricular filling volume and increased filling pressure, resulting from decreased diastolic properties and compliance of the ventricles, while the ventricular systolic function remains in the normal range. This leads to the clinical syndromes of congestion of pulmonary and systemic circulation. The pathophysiological mechanisms of HFpEF are complex and diverse. At present, there are five main mechanisms that have been deeply explored, including diastolic dysfunction, inflammation and oxidative stress/endothelial dysfunction, chronotropic incompetence and cardiac reserve dysfunction, pulmonary hypertension, and abnormal ventricular-arterial coupling. These five mechanisms are intertwined, influence and promote each other, which substantially increases the complexity of disease diagnosis and the difficulty of treatment. Consequently, in the current clinical field, the development of specific drugs for HFpEF has been challenging, and effective treatment methods remain scarce. Therefore, it is of great significance and urgency to explore the molecular mechanism of HFpEF and to find new therapeutic targets. This endeavor plays a vital role in improving the living conditions of patients, advancing the cardiovascular disease treatment technology, and reducing medical burden.

Serine/arginine splicing factor 1 (SRSF1), also known as ASF/SF2, belongs to the SR protein family. It is an indispensable and important regulator for mRNA splicing and alternative splicing. SRSF1, as a typical member of the highly conserved SR protein family, also plays a key role in maintaining genomic stability, cell viability, and cell cycle progression. In addition to its splicing function, SRSF1 can further regulate the processes of mRNA transcription, mRNA stability and protein translation. Studies have shown that in the case of overexpression, SRSF1 can promote the transformation of oncogenes in fibroblasts and epithelial cells by promoting proliferation and inhibiting apoptosis. However, the role of SRSF1 in HFpEF has not been reported yet.

### SUMMARY

In view of this, an objective of the embodiments of the present disclosure is to provide use of SRSF1 as a target in preparation of a medicament for prophylaxis and/or treatment of HFpEF. Suppressing the expression of SRSF1 gene or protein has a significant improvement effect on the HFpEF and complications thereof.

To achieve the above objective, the present disclosure provides the following technical solutions.

The present disclosure provides use of SRSF1 as a target in preparation of a medicament for prophylaxis and/or treatment of HFpEF.

As an embodiment, the SRSF1 is selected from the group consisting of an SRSF1 protein and an SRSF1-coding gene.

In some embodiments, the medicament suppresses expression of the SRSF1 protein or the SRSF1-coding gene.

As an embodiment, the medicament is one or more selected from the group consisting of the following medicaments:
1) a medicament for improving diastolic dysfunction;
2) a medicament for lowering blood pressure;
3) a medicament for alleviating pulmonary edema;
4) a medicament for improving myocardial fibrosis;
5) a medicament for alleviating myocardial hypertrophy and myocardial remodeling;
6) a medicament for improving abnormal lipid metabolism;
7) a medicament for improving abnormal glucose metabolism; and
8) a medicament for inhibiting an endothelial-to-mesenchymal transition process of a human umbilical vein endothelial cell (HUVEC).

The present disclosure further provides use of an SRSF1 inhibitor in preparation of a medicament for prophylaxis and/or treatment of HFpEF, where the SRSF1 inhibitor is selected from the group consisting of an SRSF1 gene inhibitor and an SRSF1 protein inhibitor.

As an embodiment, the SRSF1 gene inhibitor includes at least one RNA selected from the group consisting of small interfering RNA (siRNA) targeting gene SRSF1, short-hairpin (shRNA) targeting gene SRSF1 and single guide RNA (sgRNA) targeting gene SRSF1, and gene derivatives thereof.

As an embodiment, the siRNA targeting gene SRSF1 has the nucleotide sequences of SEQ ID NO: 1 and SEQ ID NO: 2.

In some embodiments, the SRSF1 protein inhibitor is selected from the group consisting of an anti-SRSF1 antibody, an SRSF1 protein-binding molecule, and an SRSF1 protein degrader.

The present disclosure further provides a medicament for prophylaxis and/or treatment of HFpEF, where the medicament includes the foregoing SRSF1 inhibitor and a pharmaceutically acceptable vehicle.

In the present disclosure, the HFpEF is selected from the group consisting of HFpEF-1, HFpEF-2, HFpEF-3, HFpEF-4, and HFpEF-5.

Compared with the prior art, embodiments of the present disclosure have the following beneficial effects.

The present disclosure determines that SRSF1 can be used as a key target for preparing a medicament for the prophylaxis and/or treatment of HFpEF. In the research process of the present disclosure, by constructing an animal model of HFpEF and conducting in-depth experiments at the cellular level, it is strongly confirmed that SRSF1 is deeply involved in and regulates the pathological development of HFpEF. In vivo modeling experiment of HFpEF was conducted on SRSF1 endothelial cell-specific knockout mice. It is indicated that suppression of SRSF1 expression may feasibly and effectively improve diastolic dysfunction and hypertension symptoms induced by HFpEF, while significantly alleviating pulmonary edema, inhibiting the progression of myocardial fibrosis and improving myocardial remodeling caused by myocardial hypertrophy, so as to fully exerting the positive effects of alleviating HF signs. Moreover, suppression of SRSF1 expression is also capable of improving the body's abnormal glucose and lipid metabolism, and significantly inhibiting the endothelial-to-mesenchymal transition process of human umbilical vein endothelial cells (HUVECs). It is further demonstrated that suppression of SRSF1 may effectively delay the progression of HFpEF, and has certain positive significance and potential value for reducing the occurrence of complications related to the disease. In the present disclosure, SRSF1 serves as a target for the prophylaxis and treatment of HFpEF. This not only opens a new path for screening highly valuable new drugs, but also provides a new target and a method for the treatment of HFpEF, holding crucial significance and value in medical research and clinical practice.

### BRIEF DESCRIPTION OF THE DRAWINGS

FIGS. 1A-1J illustrate physiological and biochemical conditions of animals after HFpEF modeling, where FIG. 1A shows representative M-mode images of mice in Chow group and HFpEF group; FIG. 1B shows left ventricular ejection fraction (LVEF) of mice in Chow group and HFpEF group; FIG. 1C shows left ventricular fractional shortening (LVFS) of mice in Chow group and HFpEF group; FIG. 1D shows representative Pulse-Wave images and Tissue Doppler images of mice in Chow group and HFpEF group; FIG. 1E shows an E/A ratio of mice in Chow group and HFpEF group; FIG. 1F shows an E/e′ ratio of mice in Chow group and HFpEF group; FIG. 1G shows left ventricular global longitudinal strain (GLS) of mice in Chow group and HFpEF group; FIG. 1H shows the maximum running distances of mice in Chow group and HFpEF group in exercise tolerance test; FIG. 1I shows systolic blood pressure (SBP) of mice in Chow group and HFpEF group; and FIG. 1J shows diastolic blood pressure (DBP) of mice in Chow group and HFpEF group, where Chow represents the control group, and HFpEF represents the model group;

FIG. 2A-2G illustrate expression of SRSF1 at the animal and cellular levels, where FIG. 2A shows mRNA levels of SRSF1 in the hearts of mice in Chow group and HFpEF group; FIG. 2B presents representative Western blot images and average data of SRSF1 in the hearts of mice in Chow group and HFpEF group; FIG. 2C shows representative immunofluorescence images showing SRSF1 and CD31 in the hearts of mice in Chow group and HFpEF group (scale, 20 μm); FIG. 2D shows mRNA levels of SRSF1 in HUVECs stimulated by high glucose for 24 h; FIG. 2E shows representative Western blot images and average data of SRSF1 in HUVECs stimulated by high glucose for 24 h; FIG. 2F shows mRNA levels of SRSF1 in HUVECs stimulated by palmitic acid for 24 h; and FIG. 2G shows representative Western blot images and average data of SRSF1 in HUVECs stimulated by palmitic acid for 24 h, where Chow represents the control group, and HFpEF represents the model group;

FIG. 3A-3K illustrate physiological and biochemical conditions of SRSF1 endothelial cell-specific knockout mice, where FIG. 3A shows representative images of PCR identification of tail DNA genomes of WT and Srsf1^{endo-/-} mice; FIG. 3B shows representative M-mode images of WT and Srsf1^{endo-/-} mice fed Chow or HFD+L-NAME; FIG. 3C shows LVEF of WT and Srsf1^{endo-/-} mice fed Chow or HFD+L-NAME; FIG. 3D shows LVFS of WT and Srsf1^{endo-/-} mice fed Chow or HFD+L-NAME; FIG. 3E shows representative Pulse Wave images and Tissue Doppler images of WT and Srsf1^{endo-/-} mice fed Chow or HFD+L-NAME; FIG. 3F shows an E/A ratio of WT and Srsf1^{endo-/-} mice fed Chow or HFD+L-NAME; FIG. 3G shows an E/e′ ratio of WT and Srsf1^{endo-/-} mice fed Chow or HFD+L-NAME; FIG. 3H shows left ventricular GLS of WT and Srsf1^{endo-/-} mice fed Chow or HFD+L-NAME; FIG. 3I shows maximum running distances of WT and Srsf1^{endo-/-} mice fed Chow or HFD+L-NAME; FIG. 3J shows SBP of WT and Srsf1^{endo-/-} mice fed Chow or HFD+L-NAME; and FIG. 3K shows DBP of WT and Srsf1^{endo-/-} mice fed Chow or HFD+L-NAME, where WT represents wild-type, Srsf1^{endo-/-} represents the SRSF1 endothelial cell specific knockout mouse, Chow represents the control group, and HFpEF represents the model group;

FIGS. 4A-4J show indexes of lung, heart tissue, and glucose and lipid metabolism in SRSF1 endothelial cell-specific knockout mice, where FIG. 4A shows lung wet/dry weight ratios of WT and Srsf1^{endo-/-} mice fed Chow or HFD+L-NAME; FIG. 4B shows heart weight/tibia length (HW/TL) ratios of WT and Srsf1^{endo-/-} mice fed Chow or HFD+L-NAME; FIG. 4C shows heart weight/body weight (HW/BW) ratios of WT and Srsf1^{endo-/-} mice fed Chow or HFD+L-NAME; FIG. 4D shows characteristic pathological staining images of heart tissues in WT and Srsf1^{endo-/-} mice fed Chow or HFD+L-NAME, top: Masson staining (scale, 50 m); medium: Sirius red staining (scale, 50 m); bottom: wheat germ agglutinin (WGA) staining (scale, 20 m); FIG. 4E shows an assessment of statistical data of myocardial fibrosis by Masson staining of heart tissue in WT and Srsf1^{endo-/-} mice fed Chow or HFD+L-NAME; FIG. 4F shows an assessment of statistical data of myocardial fibrosis by Sirius red staining of heart tissue in WT and Srsf1^{endo-/-} mice fed Chow or HFD+L-NAME; FIG. 4G shows statistical data of cardiomyocyte cross-sectional areas by WGA staining of heart tissue in WT and Srsf1^{endo-/-} mice fed Chow or HFD+L-NAME; FIG. 4H shows plasma triglyceride (TG) levels in WT and Srsf1^{endo-/-} mice fed Chow or HFD+L-NAME; FIG. 4I shows plasma total cholesterol (TC) levels in WT and Srsf1^{endo-/-} mice fed Chow or HFD+L-NAME; and FIG. 4J shows blood glucose levels (left) and areas under curve (AUC, right) of WT and Srsf1^{endo-/-} mice fed Chow or HFD+L-NAME at each time point, where WT represents wild-type, Srsf1^{endo-/-} represents the SRSF1 endothelial cell specific knockout mouse, Chow represents the control group, and HFpEF represents the model group;

FIGS. 5A-5F illustrate effects of knockdown of SRSF1 on HUVECs, where FIG. 5A shows representative Western blot images and average data of SRSF1-targeted siRNA-treated HUVECs; FIG. 5B shows mRNA levels of GLUT1, PFKFB3, and PKM2 in SRSF1-targeted siRNA-treated HUVECs; FIG. 5C shows representative Western blot images and average data of GLUT1, PFKFB3, and PKM2 in SRSF1-targeted siRNA-treated HUVECs; FIG. 5D shows glucose content in the culture medium of SRSF1-targeted siRNA-treated HUVECs; FIG. 5E shows lactate content in the culture medium of SRSF1-targeted siRNA-treated HUVECs; and FIG. 5F shows representative Western blot images and average data of α-SMA, N-cadherin, and E-cadherin in SRSF1-targeted siRNA-treated HUVECs; and

FIGS. 6A-6I illustrate effects of SRSF1 overexpression on HUVECs, where FIG. 6A shows representative Western blot images of SRSF1 in HUVECs infected with Ad-SRSF1 for 48 h; FIG. 6B shows mRNA levels of GLUT1, PFKFB3 and PKM2 in HUVECs infected with Ad-SRSF1; FIG. 6C shows representative Western blot images and average data for GLUT1, PFKFB3 and PKM2 in HUVECs infected with Ad-SRSF1; FIG. 6D shows a representative plot of extracellular acidification rate (ECAR) of HUVECs infected with Ad-SRSF1; FIG. 6E shows glycolysis levels of HUVECs infected with Ad-SRSF1; FIG. 6F shows glycolytic capacity of HUVECs infected with Ad-SRSF1; FIG. 6G shows glucose content in the medium of HUVECs infected with Ad-SRSF1; FIG. 6H shows lactate content in medium of HUVECs infected with Ad-SRSF1; and FIG. 6I shows representative Western blot images and average data for α-SMA, N-cadherin and E-cadherin in HUVECs infected with Ad-SRSF1.

### DETAILED DESCRIPTION OF THE EMBODIMENTS

Discovered is a new target SRSF1 for treating HFpEF through research on HFpEF. Thus, the present disclosure provides use of SRSF1 in preparation of a medicament for prophylaxis and/or treatment of HFpEF.

In the present disclosure, the SRSF1 is selected from the group consisting of an SRSF1 protein and an SRSF1-coding gene. In the present disclosure, the human SRSF1 gene sequence is identified by Gene ID: 6426, and the mouse SRSF1 gene sequence is identified by Gene ID: 110809. In the present disclosure, the medicament is a medicament for suppressing the expression of the SRSF1 protein or the SRSF1-coding gene.

In the present disclosure, a C57BL/6 mouse model, SRSF1 endothelial cell-specific knockout mice (SRSF1^{endo-/-}) and human umbilical vein endothelial cells (HUVECs) are used as subjects to investigate the role of SRSF1 in HFpEF. In the present disclosure, there is no particular restriction on the modeling of the C57BL/6 mouse model. A widely accepted method in the art is to establish a HFpEF model using a combination of high-fat diet (HFD) and NO-nitro-L-arginine methyl ester (L-NAME, a nitric oxide synthase inhibitor). Similarly, there is no particular restriction on the modeling method of HUVECs. A commonly known approach in the art is to construct a high glucose (HG) and high fat cell model by combined stimulation using high glucose and palmitic acid (PA). Through the model of HFpEF in mice, it is found that the left ventricular strain rate of the induced mice is significantly decreased, namely, the diastolic function of the animal heart is impaired, and there is obvious diastolic dysfunction. At the same time, the exercise tolerance of the animal decreased, which is in line with the signs of HFpEF, demonstrating that the model is established successfully. By jointly detecting the heart tissue of the animal model and the tissue of the HUVEC model, it is found that SRSF1 is highly expressed in the heart tissue and HUVECs, revealing that SRSF1 is involved in regulating the pathological process of HFpEF. In the experiment of SRSF1 endothelial cell-specific knockout mice, it is found that SRSF1 endothelial cell-specific knockout may significantly ameliorate diastolic dysfunction and hypertension signs in mice with HFpEF, and significantly improve pulmonary edema. At the same time, the staining of myocardial tissue sections has found that SRSF1 endothelial cell-specific knockout may significantly improve myocardial fibrosis, myocardial hypertrophy and myocardial remodeling. It is further found that SRSF1 endothelial cell-specific knockout also has a significant effect on the regulation of metabolism, significantly improving abnormal glucose and lipid metabolism. In the present disclosure, it is further found that knockdown of SRSF1 can inhibit the endothelial-to-mesenchymal transition process of HUVECs by interfering with the expression of SRSF1 in HUVECs, while overexpression of SRSF1 can promote the endothelial-to-mesenchymal transition process of HUVECs. In summary, SRSF1 endothelial cell-specific knockout and interference may play a role in alleviating HF, thus playing a role in the treatment of HFpEF. The findings of the present disclosure contribute to the current understanding of the pathogenesis of HFpEF, and therapeutic interventions by suppressing SRSF1 may provide a potential new treatment strategy for HFpEF.

The present disclosure provides use of an SRSF1 inhibitor in preparation of a medicament for prophylaxis and/or treatment of HFpEF. In the present disclosure, the SRSF1 inhibitor is selected from the group consisting of an SRSF1 gene inhibitor and an SRSF1 protein inhibitor. The SRSF1 gene inhibitor includes at least one RNA selected from the group consisting of siRNA targeting gene SRSF1, shRNA targeting gene SRSF1 and sgRNA targeting gene SRSF1 and gene derivatives thereof. As an embodiment, the siRNA targeting gene SRSF1 has the nucleotide sequences of SEQ ID NO: 1 and SEQ ID NO: 2. The SRSF1 gene inhibitor may further be selected from the group consisting of a nucleic acid construct and a lentivirus. The nucleic acid construct is a gene segment encoding the foregoing nucleic acid molecule, capable of expressing the foregoing nucleic acid molecule. The lentivirus is packaged by the foregoing nucleic acid construct with the help of a lentiviral packaging plasmid and a cell line. The SRSF1 protein inhibitor is selected from the group consisting of an anti-SRSF1 antibody, an SRSF1 protein-binding molecule, and an SRSF1 protein degrader. As an alternative embodiment, the SRSF1 protein inhibitor may be selected from the group consisting of labetalol and betaxolol, but is not limited thereto. The labetalol or the betaxolol binds to the SRSF1 protein, thus affecting the function of the SRSF1 protein.

The present disclosure further provides a medicament for prophylaxis and/or treatment of HFpEF, where the medicament includes the foregoing SRSF1 inhibitor and a pharmaceutically acceptable vehicle. The pharmaceutically acceptable vehicle is selected from the group consisting of buffers, excipients, stabilizers and preservatives, such as starch, lactose, magnesium stearate, water, brine, buffer, glycerin, ethanol, liposomes, lipids, proteins, protein-antibody conjugates, peptides, cellulose, nanogels or combinations thereof, but is not limited thereto, as long as the vehicle is matched with the pharmaceutical dosage form.

In this disclosure, the HFpEF is heart failure with a LVEF ≥50%. The HFpEF is selected from the group consisting of HFpEF-1, HFpEF-2, HFpEF-3, HFpEF-4, and HFpEF-5. The HFpEF-1 is a vascular disease-related HFpEF that is associated with hypertension, coronary artery disease, and coronary microvascular dysfunction; the HFpEF-2 is a cardiomyopathy-related HFpEF, and an HFpEF caused by hypertrophic cardiomyopathy, invasive cardiomyopathy such as cardiac amyloidosis and Fabry disease; the HFpEF-3 is an HFpEF associated with right heart and pulmonary artery disease, that is, an HFpEF caused by pulmonary hypertension with or without right ventricular dysfunction; the HFpEF-4 is an HFpEF associated with valvular heart disease and arrhythmia, that is, an HFpEF caused by valvular heart disease and atrial fibrillation; the HFpEF-5 is an HFpEF associated with extracardiac diseases, that is, an HFpEF caused by extracardiac diseases, mainly including metabolic diseases, such as diabetes, obesity or metabolic syndrome; diseases that frequently lead to high output state, such as anemia, liver diseases, hyperthyroidism, arteriovenous fistula, and the like; and other diseases, such as chronic kidney disease, tumor therapy, and the like.

The present disclosure has no particular restriction on the pharmaceutical dosage form, which may include tablets, injections, inhalants, granules, pills, and capsules. Various dosage forms in the present disclosure further include pharmaceutically acceptable excipients, including one or more selected from the group consisting of diluents, colorants, sweeteners, coating agents, adhesives, absorbents, disintegrants, dispersants, wetting agents, cosolvents, buffers, and surfactants. The present disclosure has no particular restriction on administration methods, and oral, intravenous, parenteral, intramuscular, subcutaneous, intraperitoneal, intranasal, rectal or topical administration may be routinely selected according to the pharmaceutical dosage form and actual needs.

In the examples of the present disclosure, all data are statistically analyzed using GraphPad Prism 9.0 software, and the data are expressed in the form of mean±standard deviation (SD). P<0.05 is defined as a significant difference.

In the following examples, all methods are conventional methods, unless otherwise specified.

All materials and reagents used in the following examples may be commercially available, unless otherwise specified.

The technical solutions provided by the present disclosure will be described in detail below with reference to accompanying drawings and examples, but they should not be construed as limiting the protection scope of the present disclosure.

### Example 1

Establishment of HFpEF model: Ten 8-week-old male C57BL/6 mice were selected and fed HFD containing 60% fat and water containing 0.5 g/L L-NAME (adjusted to pH 7.4) for 8 weeks, designated as HFpEF group (model group). Ten male C57BL/6 mice fed a chow diet were designated as Chow group (control group).

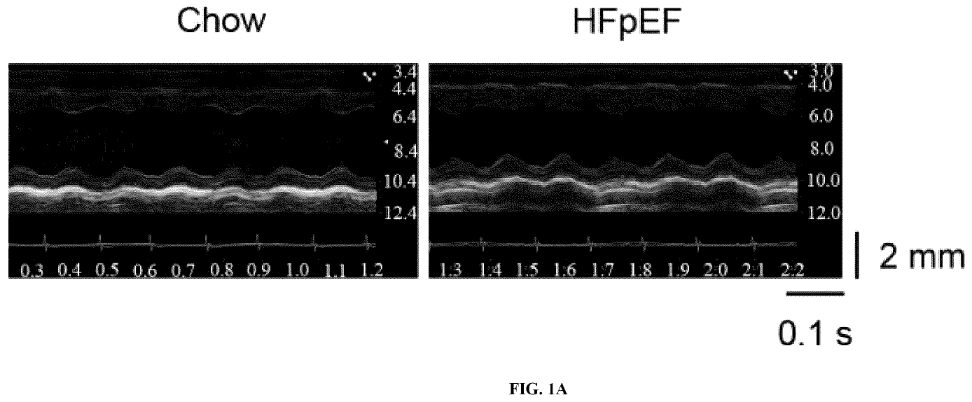

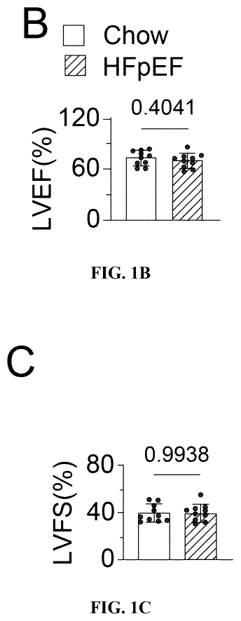

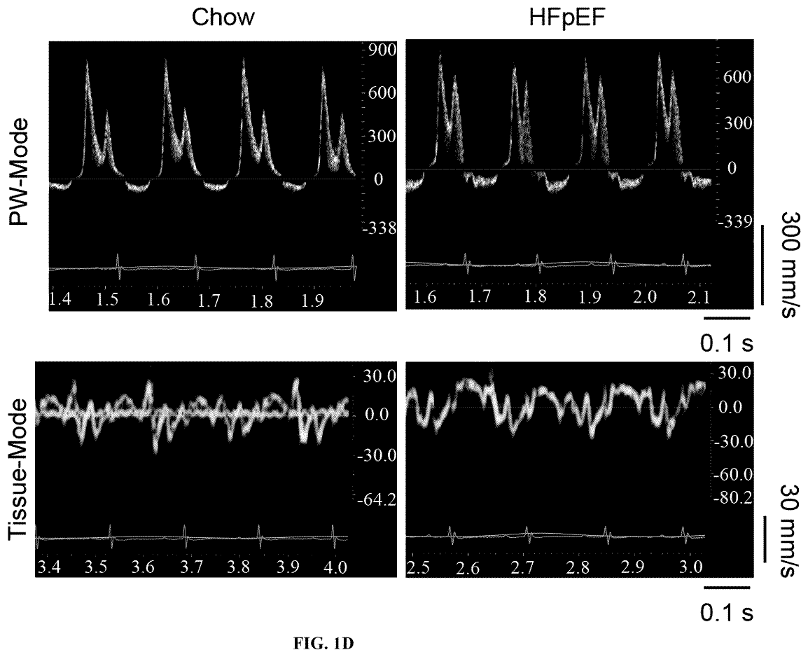

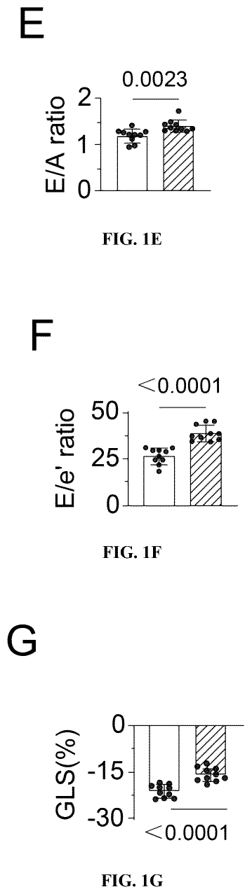

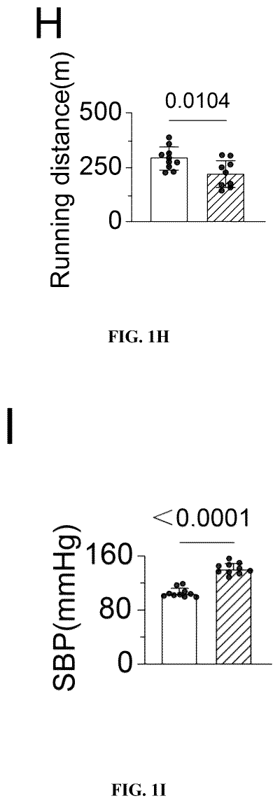

The results are shown in FIGS. 1A-1J. After 8-week feeding, the cardiac function of the mice was detected by ultrasonography. The results showed that the mice with HFpEF had no significant changes in LVEF and LVFS (%) compared with the Chow group, suggesting that the cardiac systolic function of the mice was not damaged. The maximum blood flow velocity at the mitral valve opening was determined in the Color Doppler mode and under Pulse Wave, and E-wave and A-wave were measured. It was shown that the E/A ratio was significantly increased in the mice with HFpEF. In the Tissue Doppler mode, the velocity e′ of the mitral interventricular septum-valve tissue movement was measured, and the resulting E/e′ ratio showed a significant increase in the mice with HFpEF. Left ventricular long axis views were acquired in the B-mode. The left ventricular strain rate was analyzed by speckle tracking strain imaging. The left ventricular strain rate of the mice with HFpEF decreased significantly at 8 weeks, demonstrating impaired cardiac diastolic function and obvious diastolic dysfunction. The exercise tolerance was further tested by running distances. The data showed that the running distance of the HFpEF group was significantly shorter than that of the Chow group, suggesting that the exercise tolerance of the HFpEF group was impaired. In addition, blood pressure measurements of both groups of the mice showed that the SBP and DBP of the HFpEF group were significantly higher than those of the Chow group, suggesting that the HFpEF group had signs of hypertension. The above findings demonstrated that the mouse model of HFpEF was successfully established.

### Example 2

Determination of Expression Levels of SRSF1 in Mice with HFpEF

Eight 8-week-old male C57BL/6 mice were selected and fed HFD containing 60% fat and water containing 0.5 g/L L-NAME (adjusted to pH 7.4) for 8 weeks, designated as HFpEF group (model group). Eight male C57BL/6 mice fed a chow diet were designated as Chow group (control group). The expression levels of SRSF1 in HFpEF group and Chow group were detected.

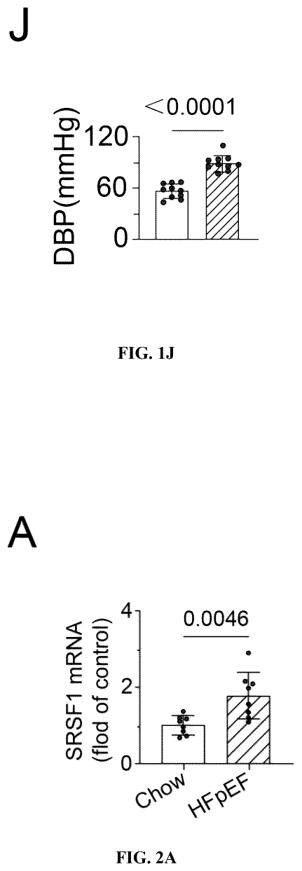

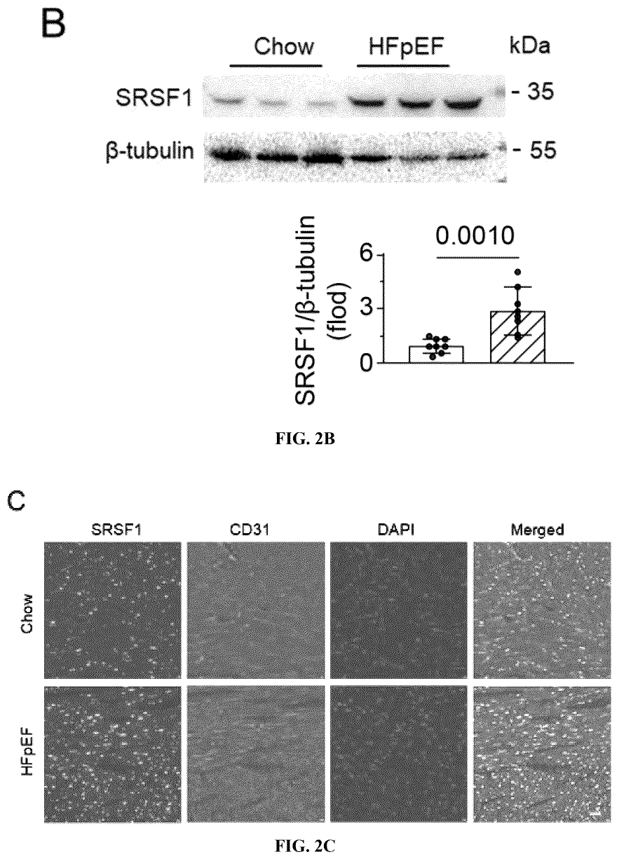

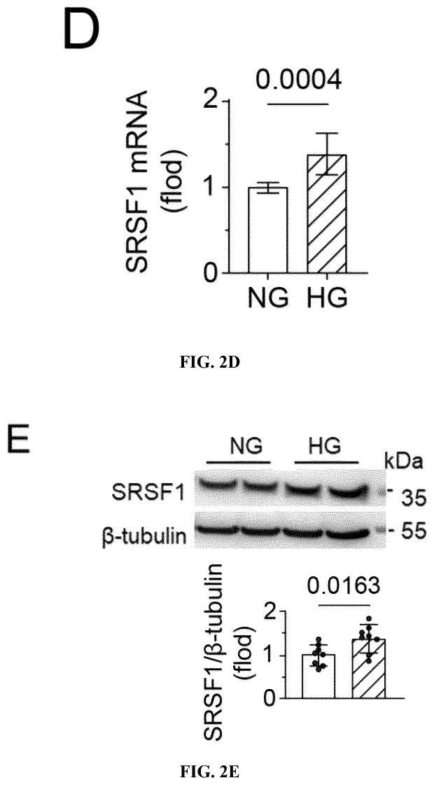

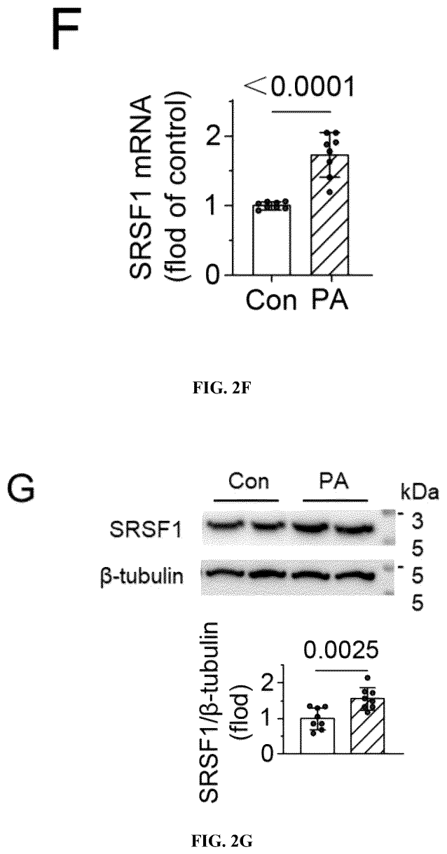

The results are shown in FIG. 2A and FIG. 2B. Compared with those in the Chow group, the mRNA and protein levels of SRSF1 in the HFpEF group were significantly increased. Immunofluorescence staining was conducted on the heart tissues of both groups of mice. The results are shown in FIG. 2C. The expression of SRSF1 in endothelial cells in the heart tissues of the mice with HFpEF was significantly increased, confirming that SRSF1 was involved in the regulation of the pathological process of HFpEF.

### Example 3

### Determination of Expression Levels of SRSF1 in High-Glucose and High-Fat HUVEC Models

HUVECs were induced in Dulbecco's Modified Eagle's Medium (DMEM) supplemented with 5.5 mmol/L glucose for 24 h as the control group (NG), and in DMEM supplemented with 33 mmol/L glucose for 24 h, designated as the high glucose treatment group (HG). A high-fat cell model was established by inducing HUVECs with 0.2 mmol/L palmitic acid for 24 h.

The expression level of SRSF1 was detected after induction of high glucose and high fat. The results are shown in FIG. 2D, FIG. 2-E, FIG. 2F and FIG. 2G. It was found that the mRNA and protein levels of SRSF1 in HUVECs were significantly increased, confirming that SRSF1 was involved in the pathological process of HFpEF.

### Example 4

### Verification of Potential Role of SRSF1 in HFpEF

Modeling of SRSF1 endothelial cell-specific knockout mice (Srsf1^{endo-/-}): Hybrid mice were obtained by hybridizing SRSF1^{flox/flox} mice provided by Professor Fu Xiangdong's team with Tie2-Cre mice provided by Beijing Viewsolid Biotechnology Co., Ltd. SRSF1 knockout mice driven by endothelial cell-specific promoter, namely Srsf1^{endo-/-} mice, were confirmed by PCR identification of mouse tail gene.

Ten male C57BL/6 mice aged 8-12 weeks and 10 Srsf1^{endo-/-} mice aged 8-12 weeks were fed HFD containing 60% fat and water containing 0.5 g/L L-NAME (adjusted to pH 7.4) for 8 weeks, serving as HFpEF group. Among them, male C57BL/6 mice and Srsf1^{endo-/-} mice were designated as HFpEF+WT group (wild type group) and HFpEF+Srsf1^{endo-/-} group, respectively. Another 10 male C57BL/6 mice aged 8-12 weeks and 10 Srsf1^{endo-/-} mice aged 8-12 weeks were fed a chow diet for 8 weeks, serving as the Chow group. The above two groups were compared and designated as WT+Chow group and Srsf1^{endo-/-}+Chow group, respectively.

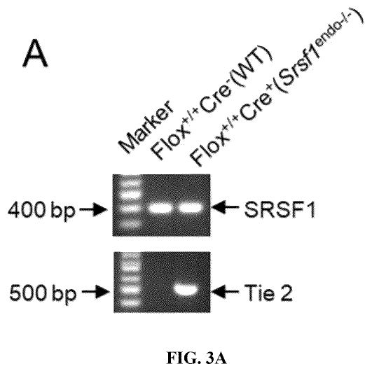

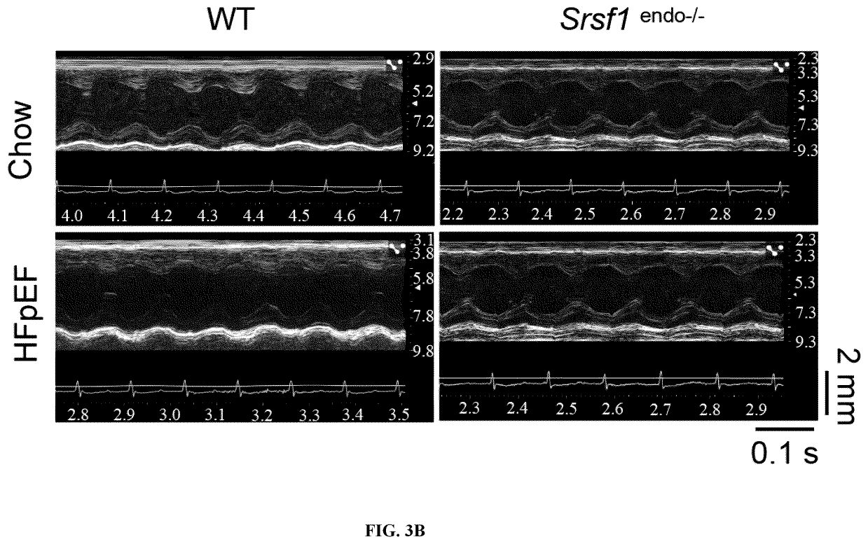

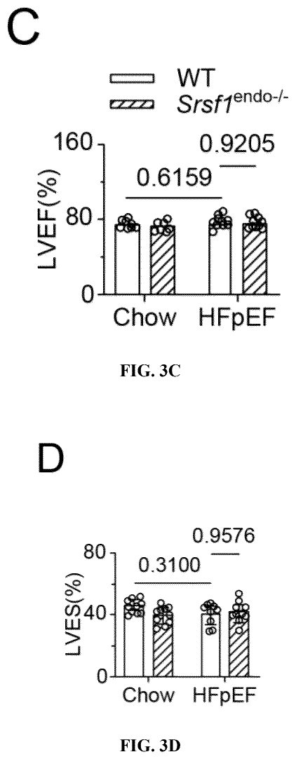

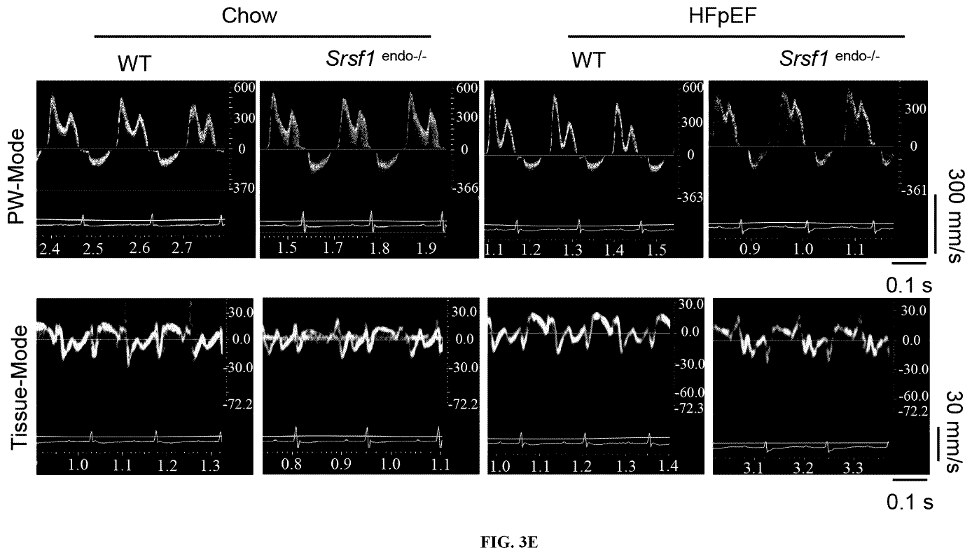

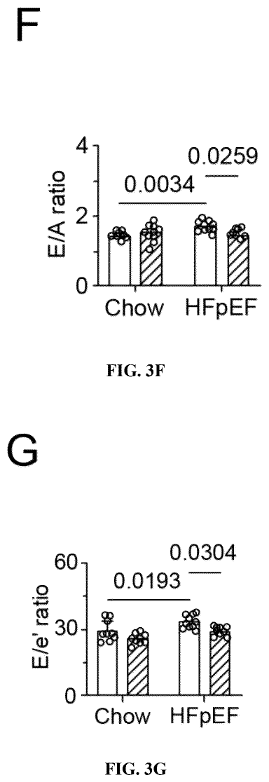

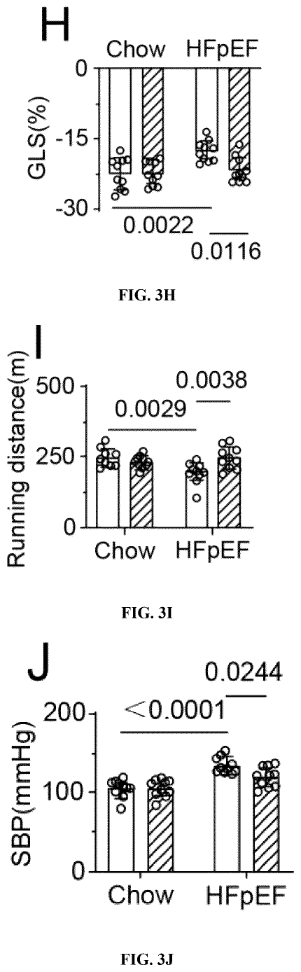

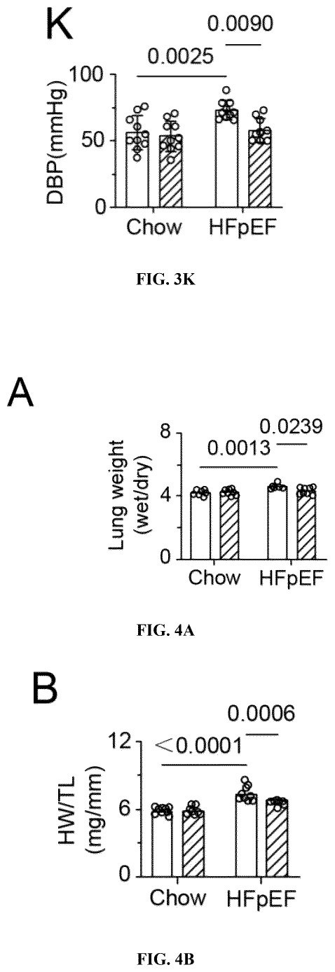

The results are shown in FIGS. 3A-3K. The cardiac function of mice was evaluated by ultrasonography. FIG. 3B, FIG. 3C and FIG. 3D showed that LVEF and LVFS in the HFpEF group showed no significant decrease compared with those in the Chow group, suggesting that the mouse heart maintained normal systolic function. The values of E, A and e′ were measured under Pulse Wave and Tissue Doppler conditions. The results of data processing and analysis showed that E/A and E/e′ of Srsf1^{endo-/-} mice in the HFpEF group were significantly lower than those in the WT group. The statistical results of left ventricular global longitudinal strain (GLS) suggested that the left ventricular strain rate of the Srsf1^{endo-/-} mice was significantly higher than that of the WT group. This suggested that SRSF1 knockout improved the impaired diastolic function of HFpE. The exercise tolerance test showed that among the mice fed high-fat+L-NAME, the maximum running distance of Srsf1^{endo-/-} mice was significantly longer than that of WT mice, further indicating that SRSF1 knockout enhanced the exercise tolerance to HFpEF. The results are shown in FIG. 3I. In addition, SRSF1 knockout significantly reduced levels of SBP and DBP in HFpEF mice. The results are shown in FIG. 3J and FIG. 3K. In summary, SRSF1 endothelial cell-specific knockout significantly ameliorated diastolic dysfunction and hypertension signs in mice with HFpEF, indicating that suppression of SRSF1 expression may improve diastolic dysfunction and lower blood pressure.

### Example 5

### Verification of Effects of SRSF1 on Heart and Lung Tissues and Glucose and Lipid Metabolism in HFpEF Group

The lung and heart tissues of each group of mice in Example 4 were sampled for detection, and the severity of pulmonary edema was evaluated by measuring and calculating the lung wet/dry weight ratio. The left ventricular myocardial tissue was stained with Masson and Sirius red stain, and the severity of myocardial fibrosis in each group was calculated statistically. At the same time, wheat germ agglutinin (WGA) staining was performed to calculate the cardiomyocyte cross-sectional area in each group and evaluate the severity of myocardial hypertrophy. The metabolic function was evaluated, and the levels of plasma TG, TC and blood glucose in mice were detected.

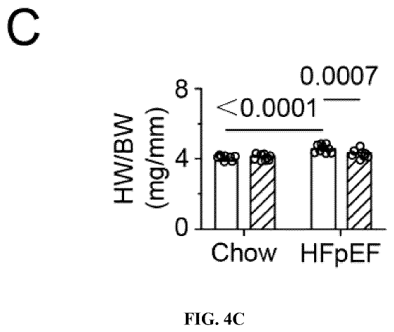

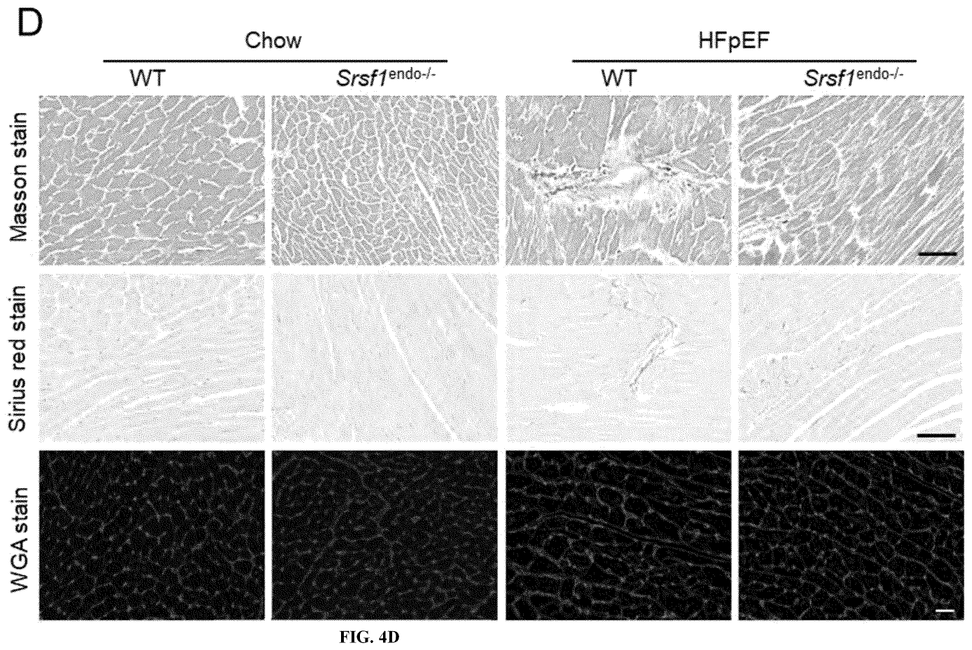

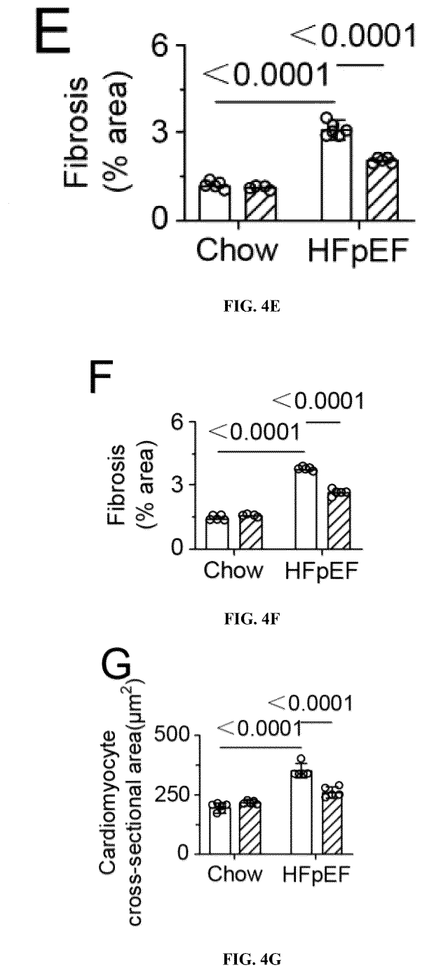

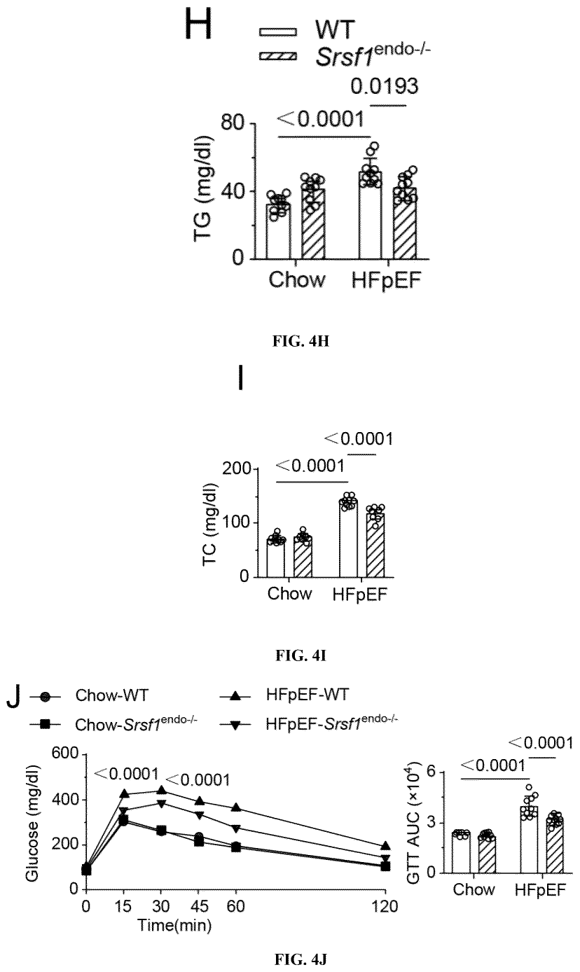

As shown in FIGS. 4A-4J, the lung wet/dry weight ratio was significantly higher in the HFpEF group than in the Chow group, showing more severe pulmonary edema, and SRSF1 endothelial cell-specific knockout could significantly improve this condition. At the same time, Srsf1^{endo-/-} mice exhibited lower heart weight/tibia length (HW/TL) and heart weight/body weight (HW/BW) ratios under high fat+L-NAME feeding conditions, suggesting that SRSF1 knockout improved myocardial hypertrophy and alleviated HF signs. SRSF1 endothelial cell-specific knockout significantly improved myocardial fibrosis and myocardial hypertrophy induced by HFD+L-NAME, manifesting as decreased collagen deposition and reduced cardiomyocyte cross-sectional area. Subsequent evaluation of the metabolic function showed consistent results, showing that SRSF1 knockout could reverse the high TG and TC levels induced by high fat+L-NAME and improve lipid metabolism. At the same time, the detection of glucose tolerance by the glucose tolerance test (GTT) showed that the mice in HFpEF group exhibited obvious abnormal glucose tolerance as compared with those in the Chow group, while the abnormality was alleviated in the Srsf1^{endo-/-} mice and the abnormal glucose metabolism was improved. In summary, SRSF1 endothelial cell-specific knockout significantly improved signs of HFpEF and abnormal glucose and lipid metabolism, indicating that suppression of SRSF1 expression may improve pulmonary edema, myocardial fibrosis, myocardial hypertrophy and remodeling, and significantly alleviate abnormal glucose and lipid metabolism.

### Example 6

### Effects of Knockdown of SRSF1 and Overexpression of SRSF1 on Metabolism and Function of Endothelial Cells

HUVECs with knockdown of SRSF1 and overexpression of SRSF1 were constructed in order to verify the effect of SRSF1 endothelial cell-specific knockout on HFpEF and further explore the mechanism in endothelial cells.

### 1. Knockdown of SRSF1

First, using RNAi Designer, two small interfering RNAs were designed for the coding region of SRSF1 to specifically knock down the expression level of SRSF1, where the sequence of SRSF1 si1 is AGACUGUGAUAUUGUGUAAAU (SEQ ID NO: 1), and the sequence of SRSF1 si2 is ATGTATGTTATGCTGATGTTTAC (SEQ ID NO: 2); using double-stranded RNA-negative control with a non-specific sequence (known as Scrambled) as a control, cells were transfected with RNAiMax for 72 h.

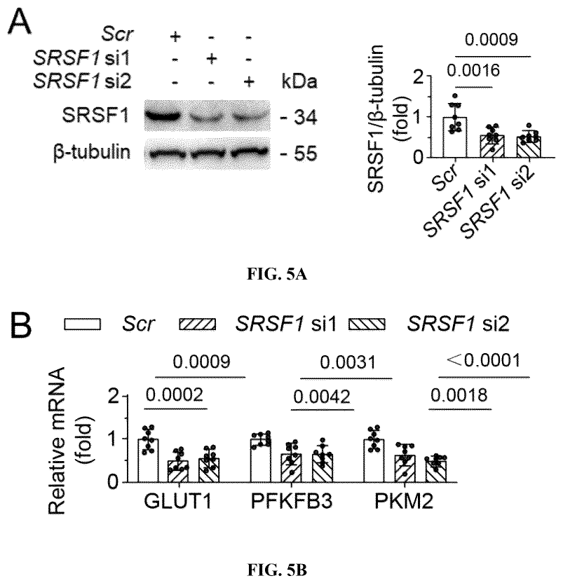

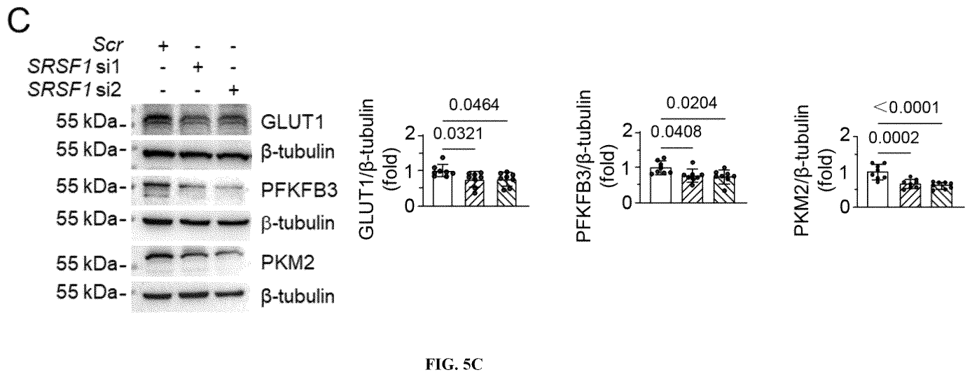

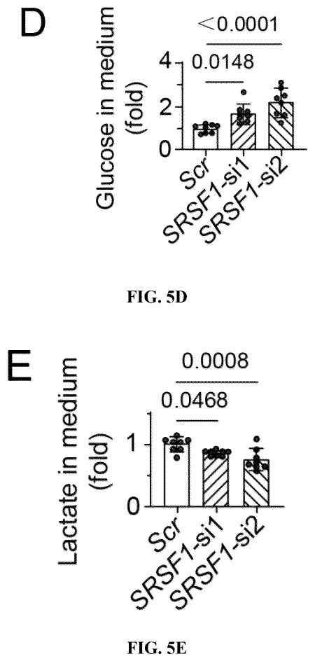

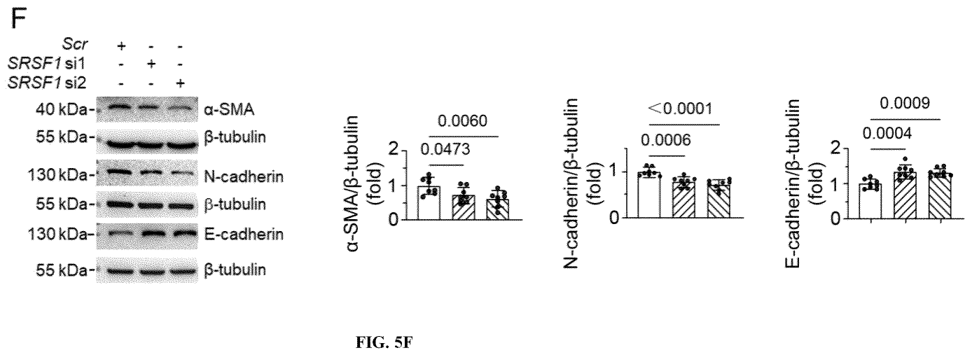

The results are shown in FIGS. 5A-5F. After transfection of HUVECs with two different SRSF1 siRNAs, the protein level of SRSF1 was significantly decreased. After knockdown of SRSF1 with SRSF1 siRNA in HUVECs, the mRNA levels of glycolytic pathway-related genes GLUT1, PFKFB3 and PKM2 were significantly decreased, and the protein levels of the above genes were also significantly lower than those of the control group. The supernatant of the medium of HUVECs with knockdown of SRSF1 was collected to determine the content of glucose and lactate. It was found that the glucose content was significantly increased and the lactate content was decreased in the supernatant with SRSF1, indicating that knockdown of SRSF1 inhibited the glycolysis level of HUVECs. Abnormal glycolytic metabolism is an important factor that causes endothelial-to-mesenchymal transition (Endo-MT). Therefore, the protein expression of endothelial-to-mesenchymal transition-related genes was detected. It was found that the protein expression of mesenchymal cell markers α-SMA and N-cadherin was significantly decreased and the protein expression of endothelial cell marker E-cadherin was promoted after SRSF1 was knocked down in HUVECs, indicating that knockdown of SRSF1 inhibited the endothelial-to-mesenchymal transition process of HUVECs.

### 2. Overexpression of SRSF1

Firstly, the adenovirus with HA tag, Ad-SRSF1-HA, was constructed by molecular biology method. The construction of the virus was completed by Beijing BAC Biological Technology Co., Ltd. The 3-gal and SRSF1 adenoviruses (Ad-SRSF1-HA) were thawed on ice, and HUVECs were taken out. The original culture medium was removed by suction pump, and serum-free Endothelial Cell Medium (ECM) was added. The viruses were diluted and added to a Petri dish or well plate, mixed well, and cultured in a CO₂ incubator for 48 h. Subsequently, the expression of SRSF1 and related proteins was detected.

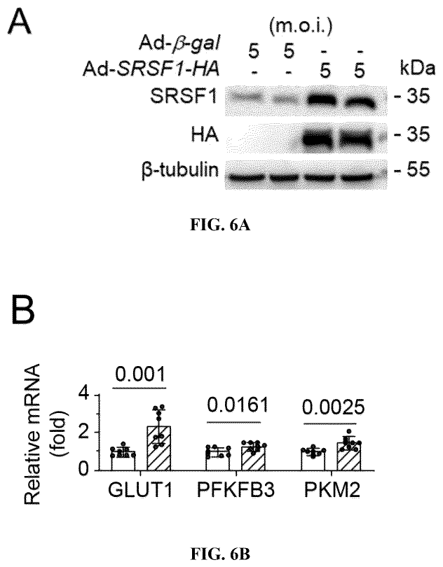

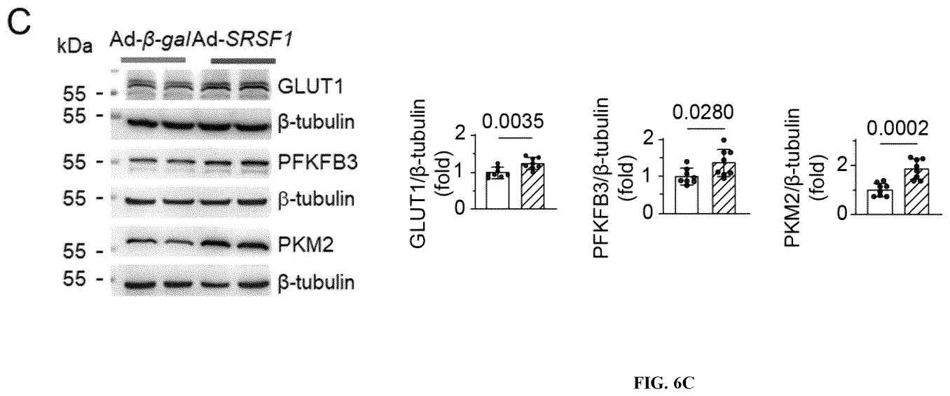

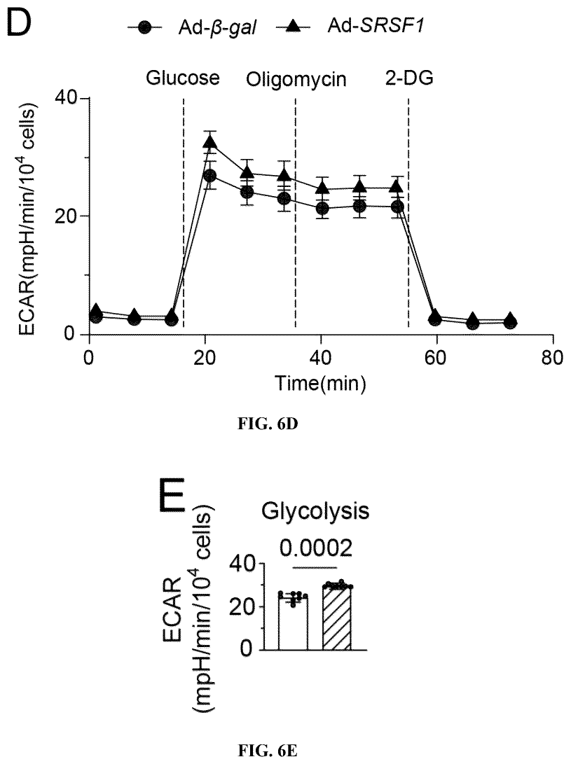

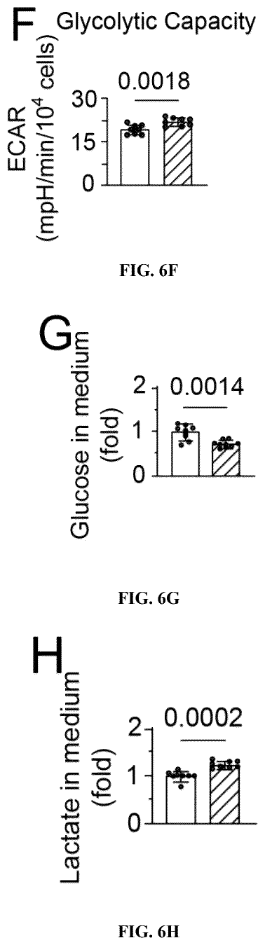

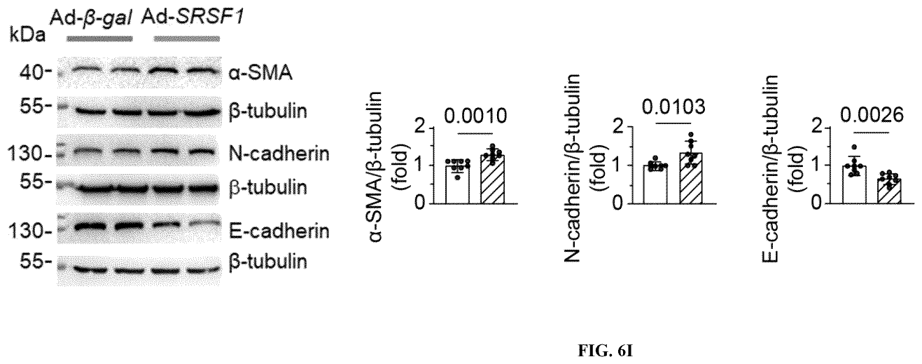

The results are shown in FIGS. 6A-6I. After successful overexpression of SRSF1 in HUVECs, the mRNA levels of glycolytic pathway-related genes GLUT1, PFKFB3 and PKM2 were significantly increased, and the protein levels of the above genes were also significantly higher than those of the control group. The evaluation of glycolysis level of the cells by Seahorse showed that overexpression of SRSF1 significantly increased the glycolysis level of HUVECs. The supernatant of the medium of HUVECs with overexpressed SRSF1 was collected to determine the content of glucose and lactate. It was found that the glucose content decreased significantly and the lactate content increased in the SRSF1-overexpressed supernatant, indicating that overexpression of SRSF1 promote the glycolysis level of HUVECs. It was also found that overexpression of SRSF1 in HUVECs could significantly promote the protein expression of mesenchymal cell markers α-SMA and N-cadherin, and suppress the protein expression of endothelial cell marker E-cadherin, indicating that overexpression of SRSF1 promoted the endothelial-to-mesenchymal transition process of HUVECs.

Conclusion: Endothelial-to-mesenchymal transition (Endo-MT) is a potential pathobiological process of disease pathogenesis. Activation of the Endo-MT mechanism enables prominent mesenchymal characteristics and excessive fibroblast proliferation, which further aggravates the ischemia and hypoxia of lung tissue and heart tissues in the body. This accelerates the progression of fibrosis, and exacerbates the pathological process of HFpEF. Therefore, suppression of SRSF1 expression may inhibit the Endo-MT process of HUVECs, thereby reducing cardiac fibrosis. This approach aims to treat HFpEF and relieve related symptoms.

The above are merely preferred embodiments of the present disclosure. It should be noted that several improvements and modifications may further be made by a person of ordinary skill in the art without departing from the principle of the present disclosure, and such improvements and modifications should also be deemed as falling within the protection scope of the present disclosure.

What is claimed is:

1. A method for preventing and/or treating heart failure with preserved ejection fraction (HFpEF), comprising administering to a subject in need thereof a medicament that suppresses serine/arginine splicing factor 1 (SRSF1).

2. The method according to claim 1, wherein the SRSF1 is selected from the group consisting of an SRSF1 protein and an SRSF1-coding gene.

3. The method according to claim 1, wherein the method suppresses the expression of the SRSF1 protein or the SRSF1-coding gene.

4. The method according to claim 3, wherein the medicament is one or more selected from the group consisting of the following medicaments:
1) a medicament for improving diastolic dysfunction;
2) a medicament for lowering blood pressure;
3) a medicament for alleviating pulmonary edema;
4) a medicament for improving myocardial fibrosis;
5) a medicament for alleviating myocardial hypertrophy and myocardial remodeling;
6) a medicament for improving abnormal lipid metabolism;
7) a medicament for improving abnormal glucose metabolism; and
8) a medicament for inhibiting an endothelial-to-mesenchymal transition process of a human umbilical vein endothelial cell (HUVEC).

5. A method for preventing and/or treating HFpEF, comprising administering to a subject in need thereof a medicament comprising an SRSF1 inhibitor, wherein the SRSF1 inhibitor is selected from the group consisting of an SRSF1 gene inhibitor and an SRSF1 protein inhibitor.

6. The method according to claim 5, wherein the SRSF1 gene inhibitor comprises at least one RNA selected from the group consisting of siRNA targeting gene SRSF1, shRNA targeting gene SRSF1 and sgRNA targeting SRSF1, and gene derivatives thereof.

7. The method according to claim 6, wherein the siRNA targeting gene SRSF1 has the nucleotide sequences of SEQ ID NO: 1 and SEQ ID NO: 2.

8. The method according to claim 5, wherein the SRSF1 protein inhibitor is selected from the group consisting of an anti-SRSF1 antibody, an SRSF1 protein-binding molecule, and an SRSF1 protein degrader.

9. A medicament for preventing and/or treating HFpEF, comprising the SRSF1 inhibitor according to claim 5 and a pharmaceutically acceptable vehicle.

10. The medicament according to claim 9, wherein the HFpEF is selected from the group consisting of HFpEF-1, HFpEF-2, HFpEF-3, HFpEF-4, and HFpEF-5.


SEQUENCE LISTING (2 sequences)

SEQ ID NO: 1 - RNA, 21 nt, synthetic construct
  source 1..21: mol_type = other RNA; note = siRNA: SRSF1 si1
agactgtgatattgtgtaaat

SEQ ID NO: 2 - RNA, 23 nt, synthetic construct
  source 1..23: mol_type = other RNA; note = siRNA: SRSF1 si2
atgtatgttatgctgatgtttac